(19) United States
(12) Patent Application Publication
(10) Pub. No.: US 2021/0139394 A1
(43) Pub. Date: May 13, 2021

(54) A PROCESS FOR THE DEHYDROGENATION OF ALKANES TO ALKENES AND IRON-BASED CATALYSTS FOR USE IN THE PROCESS

(71) Applicant: HALDOR TOPSØE A/S, Kgs. Lyngby (DK)

(72) Inventors: Poul Erik HØJLUND NIELSEN, Fredensborg (DK); Lived J. LEMUS-YEGRES, Copenhagen S (DK); Rasmus Munksgård NIELSEN, Måløv (DK)

(73) Assignee: HALDOR TOPSØE A/S, Kgs. Lyngby (DK)

(21) Appl. No.: 16/492,281

(22) PCT Filed: Mar. 2, 2018
(86) PCT No.: PCT/EP2018/055144

§ 371 Date: Sep. 9, 2019

(30) Foreign Application Priority Data
Mar. 17, 2017 (DK) PA 2017 00183

(51) Int. Cl. C07C 5/333; B01J 27/22; B01J 38/12; B01J 38/10

(52) U.S. Cl. CPC C07C 5/333; B01J 27/22; C07C 2527/22; B01J 38/10; B01J 38/12

(57) ABSTRACT

In a process for the catalytic dehydrogenation of lower alkanes to the corresponding alkenes, a regenerable catalyst comprising iron carbide supported on a carrier is used. A small amount (below 100 ppm) of a sulfur compound, such as H₂S, is added during the process.

The present invention relates to the use of iron-based catalysts in processes for the dehydrogenation of alkanes to the corresponding alkenes. More specifically, the invention relates to a process for the dehydrogenation of lower alkanes to the corresponding alkenes and a catalyst for use in the process.

Basically, the catalytic dehydrogenation of lower alkanes is a simple, but yet important reaction, which can be illustrated by the dehydrogenation of propane to propene in accordance with the reaction:

C₃H₈<->C₃H₆+H₂

With the ever growing demand for light olefins, i.e. lower aliphatic open-chain hydrocarbons having a carbon-carbon double bond, catalytic dehydrogenation is growing in importance. Especially the dehydrogenation of propane and isobutane are important reactions used commercially for the production of propylene and isobutylene, respectively. Propylene is an important basic chemical building block for plastics and resins, and the worldwide demand for propylene has been growing steadily for decades. It is expected that the demand growth for propylene will soon be equal to or even higher than that for ethylene. One of the major applications of isobutylene is as feedstock in the manufacture of methyl-tert-butyl ether (MTBE).

The process shown above is endothermic and requires about 125 kJ/mole in heat of reaction. Thus, in order to achieve a reasonable degree of conversion the dehydrogenation process is taking place at a temperature around 600° C. The dehydrogenation of isobutene is similar to that of propene in every respect, apart from requiring a slightly lower temperature.

Today there are 4 major processes for alkane dehydrogenation in commercial use. The differences between these processes primarily deal with the supply of the heat of reaction. The processes and the catalysts will be briefly described below.

### a) The Catofin Process

This process is characterized by the heat of reaction being supplied by pre-heating of the catalyst. The Catofin process is carried out in 3-8 fixed bed adiabatic reactors, using a chromium oxide/alumina catalyst containing around 20 wt % chromium oxide. The catalyst may be supplemented with an inert material having a high heat capacity, or alternatively with a material which will selectively combust or react with the hydrogen formed, the so-called heat generating material (HGM). Promoters such as potassium may be added.

The Catofin process is a very well-established process and still the dominant industrial dehydrogenation process. Since the reaction heat is supplied by the catalyst, a sequential operation is used, during which the catalyst bed is used for dehydrogenation. Then the gas is purged away, and the catalyst is being regenerated/heated and the Cr(VI) oxide reduced with hydrogen. Finally, the bed is purged with steam before another dehydrogenation.

### b) The Oleflex Process

The Oleflex process employs noble metal catalysts, especially a promoted Pt/Al₂O₃ catalyst in a reaction system of 3-4 moving bed reactors with the catalyst being continuously regenerated in a separate regeneration circuit. The heat of reaction is supplied by pre-heating the hydrocarbon stream. The noble metal catalyst is subject to slow deactivation. Thus, in the Oleflex process the catalyst moves down in the radial flow bed. In the bottom, the catalyst is transported to a regeneration reactor, where the carbon on the catalyst is burned away and the platinum is dispersed again by means of a chlorine treatment. The regenerated catalyst is recycled back into the top of the dehydrogenation reactor. The cycle time is up to one week.

The noble metal is supported on an alumina carrier, and it is stabilized by means of tin and possibly other promoters. Platinum is a good catalyst choice from a technical point of view and it forms stable alloys with tin. The main problem with this kind of catalyst is the high price, which is currently counteracted by aiming to decrease the platinum loading.

### c) The STAR Process

The STAR® process (STAR being an acronym for STeam Assisted Reforming) is a commercially established dehydrogenation technology, which has some attractive features. Steam is being used as a diluent, and the process takes place in a tubular reactor like a steam reformer placed in a furnace. The reaction heat is supplied by firing with natural gas. The catalyst is Pt supported on a ZnAl₂O₄ spinel. Zn and Pt form some very stable alloys. Some carbon deposition takes place, and the catalyst has to be regenerated every eight hours. The process is sometimes seen with a second reactor, in which a selective hydrogen combustion takes place along with further dehydrogenation. Presumably a noble metal catalyst is also being used here.

### d) The Snamprogetti-Yarzintez Process

This process is a fluid-bed version of the Catofin process, using twin fluidized beds, one each on process and regeneration duty with catalyst cycling between them. Numerous plants are in operation, e.g. in the former Soviet Union and in Saudi Arabia.

A major challenge addressed by the above processes is how to provide the reaction heat for the endothermic process. In the Catofin process (a) and the fluid-bed process (d), the heat is supplied by pre-heating of the catalyst. The catalyst used is a chromium catalyst. In the Oleflex process (b), the heat is provided by pre-heating the gas to a high temperature, whereas the STAR process (c) uses a tubular heated reactor. Both processes use a platinum-based catalyst.

In all cases (a)-(d), frequent regeneration of the catalyst is needed. In the fluid-bed and Catofin processes, this is done by re-heating the catalyst in an oxidizing atmosphere, whereas in the Oleflex process a moving bed is used, which ensures a continuous regeneration and re-heating of the catalyst, typically once a week. The catalyst consists of small spheres with a diameter of 1.6 mm, floating through radial flow beds. From the bottom of the bed it is pumped to the top of the next bed. After the fourth bed, the catalyst is sent to a regenerator where the carbon is burned off at a temperature above 480° C. In the STAR process, regeneration takes place more often.

There is an obvious interest in developing new catalysts for all the above processes, first of all because chromium and in particular chromium oxides are considered a problem. More specifically, the presence of chromium in the catalyst makes it an environmental and health hazard to handle. This is particularly so because chromium(VI)oxide, CrO₃, and related compounds of chromium in oxidation state VI are very easily formed by oxidation of the catalyst. Thus, every kind of handling of the catalyst during manufacture, transport, loading and unloading is a potential hazard, and with the increasing demand for dehydrogenation processes it is desirable to find effective, less toxic dehydrogenation catalysts. Secondly, platinum is quite expensive, and so a large capital is bound in the catalyst inventory.

Thus, the challenge here is the noble metal cost. It would therefore be desirable to replace the noble metal with a base metal, i.e. a common and inexpensive metal.

Iron is the most common and cheapest metal, and its compounds, such as iron sulfate, iron sulfide and iron carbide, are harmless. It has now turned out that iron-based catalysts can be used for all these dehydrogenation processes, provided that a small amount, more specifically below 100 ppm, of a sulfur compound is added. This compound could typically, without being limited thereto, be hydrogen sulfide.

The effects of sulfur on iron-based catalysts have been extensively studied since the recognition of its deleterious effect on the CO hydrogenation reaction. Thus, a range of sulfided iron catalysts has been synthesized by adding small amounts of Na₂S (500 to 20000 ppm) to precipitated iron (Bromfield and Coville, Appl. Catalysis A: General 186, 297-307 (1999). After calcination and reduction, the materials were subjected to syngas (H₂/CO 2:1) for up to 8 days at elevated temperature and pressure. Catalysts with low sulfide concentrations were up to four times more active in the Fischer-Tropsch reaction than a sulfur-free catalyst, while catalysts with high sulfide loadings were apparently poisoned. The results indicate that the surface area and the reducibility of the iron-based catalyst are affected by the presence of S²⁻ ions.

Various investigations of the alkane dehydrogenating ability of iron-based catalysts have been reported in the prior art. Thus, Sun et al., Chem. Eng. J. 244 145-151 (2014) have examined a sulfated iron catalyst supported on alumina at 560° C. and found an initial activity of 70 Nl/h/kg catalyst. After 6 cycles of regeneration, the activity had decreased to 50 Nl/h/kg catalyst. A study on isobutene by Wang et al., Chem. Cat. Chem. 6 2305-2314 (2014), used iron supported on silica at 560° C. and found an initial activity of 17 Nl/h/kg catalyst under conditions close to equilibrium.

The dehydrogenation of propane over alumina-supported iron-based catalysts has been investigated by Tan et al., ACS Catal. 6 5673-5683 (2016). The catalysts, having Fe/P molar ratios of 1:1, 2:1 and 3:1, were prepared via a dry impregnation method in the presence of a phosphate salt. The addition of a phosphorous source in the pre-catalyst was found to be important in obtaining a catalyst with a good performance.

Highly efficient metal sulfide catalysts for selective dehydrogenation of isobutene to isobutene have been described by Wang et al., ACS Catal. 4, 1139-1143 (2014). These metal sulfide catalysts are especially efficient in the activation of the C—H bond for isobutene dehydrogenation, and the dehydrogenation performance turned out to be better than that of the commercial catalysts Cr₂O₃/Al₂O₃ and Pt—Sn/Al₂O₃, providing a class of environmentally friendly and economical alternative catalysts for industrial applications.

U.S. Pat. No. 2,315,107 A describes a process for catalytic dehydrogenation of lower (C2-C5) alkanes to the corresponding alkenes by contacting the alkanes with an iron oxide/alumina catalyst in the presence of hydrogen sulfide.

EP 2 691 174 B1 discloses a treated catalyst for producing hydrocarbons, said catalyst comprising iron or cobalt carbide supported on a manganese oxide-based octahedral molecular sieve carrier.

Applicant's WO 2016/050583 A1 describes a process for dehydrogenation of alkanes or alkylbenzenes by using a metal sulfide catalyst in the presence of small amounts of hydrogen sulfide.

It has previously been observed that iron sulfide catalysts have a high activity and selectivity for dehydrogenation of alkanes. However, it has generally been assumed that an addition of sulfur, typically in the form of hydrogen sulfide in an amount ensuring that the catalyst is maintained as iron sulfide, would be necessary.

Now it has turned out that it is possible to use iron-based catalysts with a much smaller amount of hydrogen sulfide. In fact, experiments with X-ray powder diffraction analysis of spent catalysts have shown presence of iron carbide rather than iron sulfide, which is present at higher sulfur concentrations.

It has also turned out that iron sulfide (FeS) is by far the best metal sulfide dehydrogenation catalyst regarding selectivity because the carbon formation is very low, whereas NiS, CoS and even CuS produce much more sulfur than FeS.

Thus, iron-based catalysts can be used at low sulfur concentrations, i.e. concentrations below 100 ppm, which is an advantage since it will make the sulfur management easier. In fact, the sulfur level commonly used for process plant protection can be used. Also the regeneration of the catalyst will become easier.

So the present invention concerns a process for the catalytic dehydrogenation of lower alkanes to the corresponding alkenes according to the reaction

CₙH₂ₙ₊₂<->CₙH₂ₙ+H₂

in which n is an integer from 2 to 5, wherein the catalyst comprises a catalytically active iron compound supported on a carrier, and wherein a sulfur compound is added during the process.

It is preferred that the catalytically active iron compound is iron carbide.

The sulfur compound is typically hydrogen sulfide, added in an amount from above 0 to below 100 ppm. Even in an amount down to below 50 ppm sulfur, a dehydrogenation catalyst with a high initial activity and a very low carbon formation can be obtained.

Regeneration of the catalyst involves the following reactions:
oxidation in dilute air,
conversion of the carbide into the corresponding oxide and conversion back to the sulfide by reduction in dilute hydrogen containing hydrogen sulfide in an amount below 100 ppm, and
conversion of the sulfide into the catalytically active carbide by reaction with a carbon-containing gas.

The oxidation in dilute air is highly exothermic. Thus, an oxygen concentration of 1-2% is used.

Preferably, the oxidation is carried out at a temperature between 350 and 750° C., most preferably at a temperature between 400 and 600° C.

The invention also concerns a catalyst for use in the dehydrogenation process. Said catalyst is a regenerable catalyst comprising iron carbide supported on a carrier. The iron carbide is formed during the catalytic dehydrogenation process.

The invention is illustrated further by the examples which follow.

In the examples, reference is made to the appended FIGURE showing how an iron catalyst behaves under varying conditions.

### EXAMPLE 1

### Activity Test

The test was done using a quartz reactor placed inside a stainless steel reactor by use of the catalyst prepared in Example 2. The catalyst was heated to the process temperature using nitrogen with 2% hydrogen and 0.02% H₂S. Thus, at the start of the experiment, the iron sulfate had been converted into iron sulfide.

The influence of the propane/hydrogen ratio was studied. Experiment runs exceeding 24 hours each were done using 20 Nl/h of propane, 0.5 Nl/h of a gas containing 1% H₂S and 99% H₂ plus 5, 10 and 20 Nl/h hydrogen. The sulfur level thus corresponds to 100-200 ppm. The temperature was 620° C. and the pressure was 2 barg. The amount of propene formed declined proportionally with time. Besides, carbon was formed on the catalyst.

The amount of carbon formed was determined by treating the catalyst with dilute air and measuring the carbon dioxide formed. A subsequent reduction and sulfidation completely restored the activity. In the last sample, the carbon was determined using a LECO instrumental analysis. The selectivity was assessed by relating the amount of carbon (CO₂) formed to the amount of C₃H₆ formed on a molar basis. The results are given in Table 1 below.

TABLE 1
Selectivity results

| | Initial | | |
|---|---|---|---|
| | activity, | Deactiv. rate, % | Selectivity, Nl |
| C₃H₈/H₂ | % C₃H₈ | C₃H₆/hour | CO₂/Nl C₃H₆ |
| 4 | 13.5 | 0.21 | 0.0125 |
| 2 | 10.5 | 0.08 | 0.008 |
| 1 | 6.2 | 0.0013 | 0.002 |

### EXAMPLE 2

### Preparation of 5% Fe Catalyst

12.44 g of FeSO₄.7H₂O is dissolved in water, and the volume of the solution is adjusted to 40 ml. The solution is used to impregnate 47.5 g of a support (with a pore volume of 0.75 ml/g). The sample is rolled for 1 hour, dried overnight at 100° C. and then calcined at 600° C. for 2 hours (4 hours heating ramp). Subsequently, the sample is washed in 100 ml of a 2% K₂CO₃ solution for 1 hour (rolling board). Afterwards, the sample is washed three times with 250 ml water (1 hour each, rolling board). Finally, the sample is filtered and dried overnight at 100° C.

Data obtained by testing the prepared catalyst are given in Table 2 below.

TABLE 2

| | inlet | exit | | | | | | | |
|---|---|---|---|---|---|---|---|---|---|
| temp | flow | flow | % | % | % | % | % | % | P |
| ° C. | Nl/h | Nl/h | C₃H₈ | C₃H₆ | H₂ | CH₄ | C₂H₆ | C₂H₄ | atm |
| 630 | 45.8 | 49.6 | 44 | 9.5 | 40 | 2.6 | 2.1 | 0.9 | 3.2 |
| 620 | 30.5 | 32.9 | 44 | 9.4 | 40 | 2.5 | 2.1 | 0.7 | 3.2 |
| 610 | 30.5 | 32.6 | 48 | 8.1 | 40 | 1.9 | 1.7 | 0.4 | 3.2 |
| 600 | 30.5 | 32.4 | 52 | 7.0 | 40 | 1.1 | 1.0 | 0.3 | 3.2 |
| 620 | 61 | 65.0 | 51 | 7.0 | 41 | 1.7 | 1.1 | 0.6 | 3.5 |

### EXAMPLE 3

### Preparation of 5% Fe Catalyst

First, 133.3 g of carrier is impregnated with a solution of 26.24 g of Mg(NO₃)₂.6H₂O in 100 ml water, rolled for 1 hour, dried overnight at 100° C. and then calcined at 350° C. for 2 hours.

Then, 6.22 g of FeSO₄.7H₂O is dissolved in water, and the volume of the solution is adjusted to 19 ml. This solution is used to impregnate 23.2 g of the support previously prepared (with a pore volume of 0.75 ml/g). The sample is rolled for 1 hour and dried overnight at 100° C.

### EXAMPLE 4

### Temperature Effect

The carbon formation was measured on the catalyst prepared in Example 3 at three different temperatures using 20 or 10 Nl of Propane 10 or 5 Nl of hydrogen and 0.25 Nl of 1% H₂S in hydrogen. This corresponds to a sulfur level of 70-150 ppm. The low flow was applied at 580 and 600° C.

The results are summarized in Table 3 below.

Using thermodynamic considerations, the phase boundary between iron sulfide and iron carbide can be calculated using the reaction:

9 FeS+C₃H₈+5 H₂<->3 Fe₃C+9 H₂S

The results are given in the last column of Table 3.

TABLE 3
Temperature effect

| | Initial | | | ppm H₂S at |
|---|---|---|---|---|
| Temp. | activity, | Deactivation rate, | Selectivity, Nl | phase |
| ° C. | % C₃H₆ | % C₃H₆/hour | CO₂/Nl C₃H₆ | boundary |
| 580 | 5.8 | 0.009 | 0.0017 | 850 |
| 600 | 7.0 | 0.017 | 0.003 | 1140 |
| 620 | 8.5 | 0.061 | 0.006 | 1500 |

### EXAMPLE 5

### Preparation and Testing of 2 wt % Fe Catalyst

24.3 g of a carrier (with a pore volume of 0.75 ml/g) is placed in a beaker, and 25 ml 1-pentanol is added. The carrier is soaked in the alcohol for 10 minutes.

2.49 g of FeSO₄.7H₂O is dissolved in water, and the volume of the solution is adjusted to 20 ml. This solution is used to impregnate the pre-wet support. The sample is rolled for 1 hour, air dried for 3 hours and then dried overnight at 100° C. 10 g of the sample was placed in a quartz reactor and heated to 620° C. in a stream of H₂, N₂ and H₂S. At this condition, the state of iron is expected to be sulfidic.

Testing then took place, using 10 Nl of propane along with 5 Nl of hydrogen and 0.25 Nl of a mixture of H₂ and 1% of H₂S. After 60 hours of testing, the catalyst was regenerated using a mixture of 1% O₂ in N₂. The sulfur content is insufficient for keeping iron in the sulfide state; instead it is expected that it is carbidic as observed in previous tests.

After regeneration and resulfidation, the catalyst is tested again, this time in a mixture of 20 Nl propane and 10.25 Nl hydrogen without addition of sulfur. After 30 hours of testing, it was regenerated again and tested for 20 hours before being regenerated and resulfided.

The catalyst was tested for 15 hours in the gas containing 20 Nl propane and 10.25 Nl hydrogen. During this treatment, it deactivated from 7.7% propene to 5.8% propene. At the same time, the formation of CH₄ increased from 1.6% to 2.4%.

During the regeneration, 1.5 Nl of CO₂ was produced. This corresponds to a carbon content of 8% on the catalyst.

After regeneration and resulfidation, the catalyst was tested in a gas containing 20 Nl propane, 10 Nl hydrogen and 0.25 Nl of a mixture of 1% H₂S in H₂. During the run, there was hardly any change in the propene content, which was 7.4%. The formation of CH₄ remained at 1.5%. During the regeneration, around 0.06 Nl CO₂ was produced. This amount corresponds to a carbon content of 0.3%.

Thus it has been demonstrated that a sulfur content as low as 80 ppm in the gas is sufficient to drastically reduce the carbon formation and thereby the deactivation of the catalyst. Also the reduced formation of methane will result in a better selectivity.

### EXAMPLE 6

### Behavior of an Iron Catalyst Under Various Conditions

The experiments are typically run at a propane/hydrogen ratio of 2 with a gas containing 200 ppm H₂S and an SV of 2000. The catalyst was made by impregnation of a spherical alumina carrier with iron sulfate. It is observed that the propene content in the exit gas rises to around 11% and then falls slowly due to carbon formation which leads to clogging of the pore system.

After about 20 hours, the catalyst is regenerated with dilute air, i.e. 1-2% O₂ in N₂, and the content of CO₂ is measured. That is the black top in the FIGURE.

No matter to which iron compound the catalyst may have ended up during the dehydrogenation process, be it carbide or sulfide, then it has been converted to oxygen during the regeneration. Alternatively, by regenerating at lower temperatures, the sulfide can be converted to sulfate, but at ^{˜}620° C. iron oxide is formed. This iron oxide must be activated by a reduction. The reduction after ^{˜}20 hours takes place in a gas mixture consisting of 16% H₂ and 0.16% H₂S, the rest being N₂. The reduction itself only takes ^{˜}1 hour. Then, shifting to the reaction mixture, the reaction is run for ^{˜}7 hours each at SV 4000, 2000 and 1000, respectively.

During the subsequent regeneration, 0.54 Nl CO₂ is formed after 45 hours. The experiment is repeated at 600° C. In this case, only 0.16 Nl CO₂ has been formed after 70 hours. When the catalyst is subsequently regenerated and tested under standard conditions (SV 2000), a very high initial activity of 12%, declining to 10.3% in 85 hours, is seen. The catalyst is regenerated, and 0.19 Nl CO₂ is formed. After reduction with a gas without H₂S, the catalyst is tested under standard conditions for 92-100 hours. This time, a much lower initial activity which increases, is observed. Furthermore, formation of methane is seen and, in the subsequent regeneration, much more CO₂ (2.5 Nl in 100-105 hours) is observed. The catalyst is not reduced this time, but directly started under standard conditions after regeneration.

Again, a low initial activity and a large degree of methane formation can be seen. The amount of CO₂ formed is 4.4 Nl corresponding to 2.4 g carbon on the catalyst, i.e. slightly above 20 wt %.

Thus it has been demonstrated that the presence of even very small amounts of sulfur content, typically down to ^{˜}50 ppm, leads to a catalyst with a high initial activity and a very limited tendency to carbon formation.

1. A process for the catalytic dehydrogenation of lower alkanes to the corresponding alkenes according to the reaction
CₙH₂ₙ₊₂<->CₙH₂ₙ+H₂
in which n is an integer from 2 to 5, wherein the catalyst comprises a catalytically active iron compound supported on a carrier, and wherein a sulfur compound is added during the process.

2. The process according to claim 1, wherein the catalytically active iron compound is iron carbide.

3. The process according to claim 1, wherein the sulfur compound is hydrogen sulfide, which is added in an amount from above 0 to below 100 ppm.

4. The process according to claim 3, wherein the hydrogen sulfide is added in an amount from above 0 to below 50 ppm.

5. A catalyst for use in the catalytic dehydrogenation of lower alkanes to the corresponding alkenes according to claim 1, which is a regenerable catalyst that comprises iron carbide supported on a carrier, said iron carbide being formed during the catalytic dehydrogenation process.

6. The catalyst according to claim 5, wherein the steps for regeneration comprise
oxidation in dilute air,
conversion of the carbide into the corresponding oxide and conversion back to the sulfide by reduction in dilute hydrogen containing hydrogen sulfide in an amount below 100 ppm, and
conversion of the sulfide into the catalytically active carbide by reaction with a carbon-containing gas.

7. The catalyst according to claim 6, wherein the carbon-containing gas is the reaction mixture for dehydrogenation.
